(19) United States
(12) Patent Application Publication
(10) Pub. No.: US 2010/0280271 A1
(43) Pub. Date: Nov. 4, 2010

(54) PROCESS FOR THE PREPARATION OF AROMATIC AMINES

(75) Inventors: Knut Sommer, Krefeld (DE); Karl-Heinz Wilke, Moers (DE); Peter Lehner, Mulheim/Ruhr (DE); Franz-Ulrich Gehlen, Krefeld (DE); Leslaw Mleczko, Dormagen (DE); Stephan Schubert, Baytown, TX (US); Rainer Bellinghausen, Odenthal (DE); Evin Hizaler Hoffmann, Koln (DE)

Correspondence Address: BAYER MATERIAL SCIENCE LLC, 100 BAYER ROAD, PITTSBURGH, PA, 15205, US

(73) Assignee: Bayer MaterialScience AG, Leverkusen (DE)

(21) Appl. No.: 12/765,953

(22) Filed: Apr. 23, 2010

(30) Foreign Application Priority Data
Apr. 29, 2009 (DE) 102009019436.3

(51) Int. Cl. C07C 209/36

(52) U.S. Cl. 564/423

(57) ABSTRACT

Nitroaromatic compounds are hydrogenated in the gas phase to form aromatic amines with hydrogen in the presence of one or more catalysts arranged in stationary or virtually stationary beds in a reactor. In this process, the catalyst in the reactor is at least partly replaced continuously or at periodic intervals. At least 10% of the catalyst is replaced within each 20 day interval subsequent to start up of the reaction.

### BACKGROUND OF THE INVENTION

The present invention relates to a process for the hydrogenation of nitroaromatics to aromatic amines in the gas phase with hydrogen using a catalyst arranged in stationary or virtually stationary beds in a reactor. In this process, at least a portion of the catalyst in the reactor is replaced continuously or at periodic intervals with at least 10% of the catalyst being replaced within 20 days.

Aromatic amines are important intermediate products which must be available inexpensively and in large amounts. Aniline in particular is of great importance as an intermediate product in the preparation of di- and polyisocyanates of the diphenylmethane series (hereinafter referred to as “MDI”).

According to the prior art, MDI is obtained from the corresponding di- and polyamines, generally by phosgenation. The di- and polyamines of the diphenylmethane series (hereinafter referred to as “MDA”) are prepared by reaction of aniline with formaldehyde. Aniline in turn is usually produced on a large industrial scale by hydrogenation of nitrobenzene. The latter is obtained by nitration of benzene, so that the entire process chain can be shown in simplified form as follows:

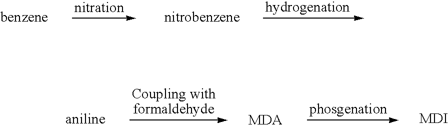

Commercially available benzene may be contaminated to a greater or lesser degree, depending on the source. Typical impurities are other aromatics, in particular toluene and xylene, which each can be present in benzene of the current purity in amounts of up to 0.05% by weight. Other typical impurities present in benzene are nonaromatic organic compounds, which can make up an amount of up to 0.07% by weight in total. Cyclohexane (up to 0.03% by weight) and methylcycohexane (up to 0.02% by weight) are to be mentioned here in particular. In the concentrations mentioned, the impurities described above either do not interfere at all or interfere only slightly in the subsequent steps in the MDI process chain, for example by making processing of the waste water and waste air in the nitrobenzene process minimally more difficult due to nonaromatic organic substances in the benzene. An expensive purification of the benzene for use in the MDI process chain would therefore be disproportionate and can be omitted.

Because of the great industrial importance of di- and polyisocyanates of the diphenylmethane series, installations with very high capacities must be built for the hydrogenation of nitrobenzene to aniline.

The hydrogenation of nitroaromatics is a highly exothermic reaction. For example, at 200° C. approx. 488 kJ mol⁻¹ are released in the hydrogenation of nitroxylene to xylidine. Approximately 544 kJ mol⁻¹ are released in the hydrogenation of nitrobenzene to aniline. The removal and use of the heat of reaction is important in carrying out processes for the hydrogenation of nitroaromatics both from the ecological and from the economic aspect.

Thus, in an established procedure, the catalyst is operated as a fluidized, heat-stabilized bed (DE-B-1 114 820). Effective removal of heat in this procedure is problematic due to a non-uniform dwell time distribution (nitrobenzene breakthrough) and abrasion of the catalyst. The patent DE-B-1 133 394 also teaches that the procedure is conducted under pressure to prolong the life of the catalysts. In a more recent approach, a fluidized catalyst bed (WO 2008/034770 A1) is used in a reactor with baffles that distribute the fluidized bed into a plurality of horizontally and a plurality of vertically arranged cells. Mass transfer and therefore the conversion are said to be improved but the reactor construction is considerably more complicated.

Narrow dwell time distribution and low abrasion of the catalyst can in principle be realized in reactors in which the bulk catalyst is stationary during the hydrogenation process (hereinafter referred to as a “fixed bed”).

Two fixed bed reactor types are often employed. One type used is a tube bundle reactor having a cooling circulation for thermostatic control of the catalyst bed(s) (so-called “isothermal procedure”). (See, for example, DE-OS 2 201 528.) In the second type, the reactor constructions contain only bulk catalysts on or between simple support grids and/or metal screens and have no system for thermal economy in the reactor, i.e. measures for thermostatic control of the catalyst bed (for example, by means of a heat transfer oil). In this second type of reactor, the reaction enthalpy is reflected quantitatively in the temperature difference between the educt and product gas stream (up to possibly unavoidable heat losses (so-called “adiabatic procedure”)). The prior art is described below with examples of both procedures.

GB 1,452,466 discloses a process for hydrogenation of nitrobenzene in which an adiabatic reactor is connected downstream of an isothermal reactor. In this process, the majority of the nitrobenzene is reacted in a thermostatically controlled tube bundle reactor. Only the hydrogenation of the residual content of nitrobenzene is carried out with a relatively small excess of hydrogen (less than 30:1) in an adiabatic reactor.

DE-OS 1 809 711 is concerned with uniform introduction of liquid nitro group containing compounds into a hot gas stream by atomization, preferably at constricted points directly upstream of the reactor. The construction of the reactor is not discussed in DE-OS 1 809 711.

DE-OS 3 636 984 describes a process for coupled production of nitro- and dinitroaromatics from the corresponding hydrocarbons by nitration and subsequent hydrogenation thereof. The hydrogenation is carried out in the gas phase at temperatures of between 176° C. and 343.5° C. An apparatus for the gas phase hydrogenation which essentially comprises two reactors connected in series with intermediate cooling and intermediate feeding in of educt is described. The size and construction of these reactors is not discussed.

In the publications mentioned above, copper catalysts operated under low loadings and at a low temperature level are employed. This results in low space/time yields.

In addition to the copper catalysts mentioned, numerous others arc described as being useful for the gas phase hydrogenation of nitroaromatics. They have been described in many publications and include as hydrogenation-active elements Pd, Pt, Ru, Fe, Co, Ni, Mn, Re, Cr, Mo, V, Pb, Ti, Sn, Dy, Zn, Cd, Ba, Cu, Ag, Au and compounds thereof, in some cases as oxides, sulfides or selenides and also in the form of a Raney alloy and on supports, such as Al₂O₃, Fe₂O₃/Al₂O₃, SiO₂, silicates, charcoal. TiO₂, and Cr₂O₃.

DE-A-2 244 401 and DE-A-2 849 002 describe palladium catalysts on aluminum oxide supports, which are operated as stationary bulk catalysts in heat exchanger tubes under normal pressure under loadings of less than 1 g_{nitroaromatic}/[ml_{catalyst}·h] with low hydrogen/nitrobenzene ratios.

DE 4 039 026 A1 describes palladium catalysts on graphitic supports which are operated under similar conditions to the palladium catalysts on aluminum oxide.

In each of these process variants, the large amount of heat of reaction produced must be removed from an industrial reactor via a heat transfer system.

DE 196 51 688 A1 describes a process for the preparation of aromatic amines in which the specific loading of the catalyst with aromatic nitro compound is increased continuously or stepwise to values of up to 5.0 kg_{nitro compound}/(l_{catalyst}·h). This results in high space/time yields. In particular embodiments, the bulk catalysts are diluted by inert packing and optionally have activity gradients. This disclosure describes hydrogenations in thermostatically controlled reactors at relatively low hydrogen excesses. The positive effects of the continuous or stepwise increase in the load and of dilution of the bulk catalyst, however, are not the consequence of the specific reactor type or of the hydrogen excess. The teaching from DE 196 51 688 A1 can therefore also be applied to an adiabatic process procedure with high hydrogen excesses.

EP 0 696 573 B1, EP 0 696 574 B1, EP 0 748 789 B1, EP 0 748 790 B1 and EP 1 882 681 A1 are directed to processes carried out under purely adiabatic conditions.

EP 0696574 B1 describes a process for the preparation of aromatic amines in which a gas mixture consisting of nitroaromatics and hydrogen flows to the catalyst under adiabatic conditions in a quite general manner.

In the processes described in EP 0 696 573 B1, EP 0 748 789 B1, EP 0 748 790 B1 and EP 1 882 681 A1, certain advantages are achieved in each case by changing various parameters.

EP 0 696 573 B1 describes the advantage of particularly high selectivities if the nitroaromatic to be reacted is also passed over the catalyst with a large amount of the aromatic amine formed during the reaction and a large amount of water, as well as with hydrogen. In this procedure, at least 2 mol of amino groups and 4 mol of water are present per mol of nitro group in each catalyst volume. The catalysts described are the same as in EP 0 696 574 B1. A disadvantage of this procedure is that large amounts of compounds which are in principle dispensable for the actual reaction, namely water and amine, have to be constantly circulated. In particular, the constant recycling of at least 2 equivalents of the amine formed, that is to say the valuable product of the process, is a great disadvantage, since the amine prepared is thereby severely exposed to heat several times.

The patents EP 0 748 789 B1 and EP 0 748 790 B1 teach that advantages are achieved merely by using specific catalyst systems.

Palladium catalysts on graphite or graphite-containing coke with a palladium content of >1.5 and ≦7% by weight are disclosed in EP 0 748 789 B1. The advantage attributed to these catalysts is their exceptionally long service life compared to all of the catalysts described earlier. The immensely high cost of the catalyst undeniably associated with the high palladium concentration is a disadvantage of this process. The patent does not discuss whether the high catalyst costs for the large amounts of palladium required for large-scale industrial use is justified by the long service life of those catalysts.

Palladium-lead catalysts on graphite or graphite-containing coke with a palladium content of >0.001 to 7% by weight are disclosed in EP 748 790 B1. The advantage attributed to these catalysts is their higher selectivity compared with analogous catalysts that do not include lead. In all the examples described in this patent, catalysts with 2% by weight of palladium were employed, so that the disadvantage of high catalyst costs also has the full effect in this case.

The patent application EP 1 882 681 A1 describes advantages which are achieved by the educt gas stream already containing significant amounts of water at the start of the hydrogenation, but at most small amounts, originating from the circulating gas stream, of the aromatic amine formed. Improvements in the service life are achieved by this means. The application also teaches an improvement in selectivity by feeding in nitrogen. This procedure also still has disadvantages. The production must be interrupted at regular intervals for regeneration of the catalyst. In addition to the loss of production undeniably associated with this, a further and even more serious disadvantage is to be seen in the fact that each new production cycle starts at a low level of selectivity. Selectivity values of >99% are already achieved after a short time only if considerable amounts of nitrogen are fed in (EP 1 882 681 A1, p. 10, paragraph [0081]), which causes high costs. Completely dispensing with nitrogen makes working up difficult, because the quality of the crude amine coming from the process is subject to wide variations in cycles under these conditions.

The publications mentioned so far either are not concerned with the problems of achieving a selectivity over the entire running time of a production cycle at as high a level as possible subject as far as possible to only very slight variations, or they merely offer expensive and therefore uneconomical solutions, such as the feeding in of very large amounts of nitrogen at the start of the hydrogenation which is the teaching of EP 1 882 681 A1.

A general improvement in the selectivity and prolonging of the catalyst service life can be achieved in principle by using catalyst beds which are arranged in flat layers and to which educt gas flows perpendicularly (so-called “radial reactors”), as is the teaching in DE 42 07 905 A1 (p. 6, 1. 61 et seq.). This application describes thermostatically controlled reactors. An adiabatically operated fixed bed process for hydrogenation of nitroaromatics (EP 0 696 574 B1) can, of course, also be carried out in radial reactors. However, such a procedure alone does not prevent the wide cyclic variations in selectivity.

Significant cyclic variations in selectivity are always a problem in principle if the activity (and therefore as a rule also the selectivity) of a catalyst at the start of a production cycle differs very widely from that at the end of the production cycle. In the context of this invention, “activity” is understood as meaning the ability of the catalyst to promote reaction of the nitroaromatics for as long as possible and as completely as possible. At the start of a production cycle, the catalyst has its highest activity and lowest selectivity. In the course of a production cycle, the activity then decreases, for example as a result of slow coking and/or sintering of the catalytically active metals, and the selectivity increases. When the conversion falls to values which are no longer acceptable, the production is interrupted and the catalyst is regenerated. In the next production cycle, the catalyst is initially present in a state which is the same as or at least similar to that of fresh catalyst, that is, it has a high activity and low selectivity. The catalyst thus passes through a broad ageing process in each production cycle. The dependency of the selectivity of the catalyst in a given incremental volume of the catalyst bed on the running time is therefore very high, and the instantaneous selectivity measured at a given point in time can differ significantly from the selectivity averaged over an entire production cycle. It would therefore appear that the problem of the wide variations in selectivity can be reduced by consciously regenerating “less well” catalyst (e.g., for a shorter time and/or at a lower temperature than would be optimum in the sense of maximum activity) after the end of a production cycle. This has the consequence that at the start of the hydrogenation in the next production cycle, the catalyst present in the fixed bed reactor behaves differently than a fresh catalyst of the same catalyst system, so that better selectivities can be achieved from the beginning than with completely (in the sense of maximum activity) regenerated catalyst, although at the expense of the service life. However, the discovery of those regeneration conditions which render possible an acceptable compromise between increased selectivity and reduced service life requires a high outlay.

Interruptions in the production process as a result of catalyst regeneration can in principle be avoided, and the problem of wide cyclic variations in selectivity can therefore also be at least somewhat reduced if spent catalyst is removed and fresh or regenerated catalyst is fed in continuously or periodically. This is possible in principle in the hydrogenation of aromatic nitro compounds with a fluidized bed process, as is the teaching in the patent specification DE-B-1 114 820 (column 2, 1.31 to 35) already mentioned and also CN-A-101 016 247, but does not solve the above-mentioned fundamental problems of this type of process.

There arc reactors which allow a replacement of catalyst without interruption of the production process and in which the catalyst is not set in fluidized motion but “flows” through suitable sluice systems under gravity (so-called “migrating bed reactors”). In this type of reactor, the bulk catalyst can be moved during the reaction, i.e., it is not to be regarded as completely stationary as in the fixed bed systems described above. The catalyst is nevertheless in the form of a bulk catalyst and is not fluidized. This intermediate position between a stationary bulk catalyst (fixed bed) and a fluidized catalyst bed, i.e. a bed kept permanently in fluidized motion (fluidized bed), is hereinafter referred to as “virtually stationary” or “migrating bed”.

The fundamental mode of functioning of such reactors has been known for a long time. Suitable systems for gas phase processes were described in the '30s of the last century (See U.S. Pat. No. 1,982,099 and U.S. Pat. No. 1,995,293.). Migrating bed reactors are currently employed in the petrochemical industry in particular, as a large number of patent applications indicate. These are refining processes for hydrocarbons which also frequently employ hydrogen.

U.S. Pat. No. 3,647,680 describes a process for the continuous operation of a reforming regeneration process, wherein a mixture of hydrocarbons and hydrogen flows laterally through a migrating catalyst bed (so-called “radial migrating bed reactor”) and wherein spent catalyst can be regenerated and fed back into the process without interrupting the process.

U.S. Pat. No. 4,133,743 is also directed to the reaction of hydrocarbons with hydrogen in a radial migrating bed reactor. Hydroreforming processes and reactions which lead to aromatic hydrocarbons are mentioned explicitly. At least two reactors are connected in series with the catalyst removed from one reactor being fed to the next. A regeneration of the catalyst takes place only after the last reactor. This disclosed process makes it possible to establish a relatively constant catalyst activity without having to interrupt the process.

U.S. Pat. No. 4,188,283 describes a start-up procedure for the hydrogenation of olefinic materials in which the feed and removal amounts or rates of the catalyst are established.

CN-A-1454970 addresses the problem of adhesion/gluing of the catalyst and a more uniform axial distribution of temperature. Hydrogen is employed in a deficit (molar ratio of H₂:hydrocarbons=1:3).

CN-A-1 333 084 describes the incorporation of an additional baffle plate in the lower part of the catalyst bed to even out transportation of the catalyst.

US-A-2006/0063957 describes the use of a radial migrating bed reactor for the preparation of propylene. The differences in reactivity within the moving catalyst bed are reduced by only partly regenerating the catalyst removed and feeding it back in a mixture with non-regenerated catalyst.

Migrating bed reactors with a large number of reaction zones within one reactor (so-called “multiple-stage reactor”) arc also known. See, e.g., U.S. Pat. No. 3,706,536 and EP-A-0 154 492.

Carrying out of reactions with extreme reaction enthalpy, such as the hydrogenation of aromatic nitro compounds, is not the subject matter of the applications mentioned for migrating bed reactors. Very highly exo- or endothermic reactions are also not easy to control in migrating bed reactors, and as is the case with the fixed bed systems, there has been no lack of attempts to make the transfer of heat controllable.

US 2006/0122446 A1 describes specific axial or radial reactors with moving catalyst beds for reactions with a high reaction enthalpy. The reactors described are divided into two reaction zones. The differences in reactivity between the two zones are balanced by admixing fresh or regenerated catalyst before each zone. The reactor construction is therefore relatively complicated. Further, in certain embodiments, an integrated heat exchanger is provided between the two reaction zones which suggest that in the case of reactions of an extremely highly exothermic character, thermostatic control cannot be dispensed with. Thermostatic control of reactors can be problematic in fixed bed systems if the production scale is very large. In migrating bed systems, thermostatic control of reactors is not impossible, as this application shows, but is of course even more complicated than in fixed bed systems.

In US 2006/0115387 A1, an integrated heat exchanger is provided in all embodiments.

### SUMMARY OF THE INVENTION

The object of the present invention was therefore to provide a process for the preparation of aromatic amines in which the crude amine can be prepared with a very high selectivity subject to only slight variations.

It has been possible to achieve this objective by a process for the hydrogenation of nitroaromatics to aromatic amines in the gas phase on catalysts arranged in stationary or virtually stationary beds by continuous or periodic replacement of at least some of the catalyst employed. The activity of the catalyst actively involved in the hydrogenation process at a given point in time is adjusted so that formation of the aromatic amine with a high selectivity which is subject to only slight variations is rendered possible.

In preferred embodiments of the present invention, the activity of the catalyst actively involved in the hydrogenation process and the selectivity with which the desired aromatic amine is formed is kept within narrow limits during the entire hydrogenation process.

### BRIEF DESCRIPTION OF THE DRAWINGS

FIG. 1a shows in diagram form a longitudinal section of a reactor suitable for conducting the process of the present invention.

FIG. 1b shows in diagram form a transverse section of a reactor suitable for conducting the process of the present invention.

FIG. 2 shows in block diagram form the process steps in one embodiment of the process of the present invention.

FIG. 3 shows in block diagram form the process steps of an embodiment of the process of the present invention which employs three reactors.

FIG. 4 is a graph on which the selectivities of the amine produced in Examples 3 and 4 are plotted against the point in time when the selectivities were determined.

### DETAILED DESCRIPTION OF THE INVENTION

The present invention relates to a process for the preparation of aromatic amine(s) of the formula

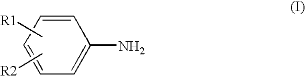

in which

R1 and R2 independently of each other represent hydrogen, a methyl or an ethyl group, wherein R1 can additionally represent an amino group,

by hydrogenation of nitroaromatic(s) of the formula

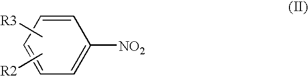

in which

R2 and R3 independently of each other represent hydrogen, a methyl or an ethyl group, wherein R3 can additionally represent a nitro group, in the gas phase with hydrogen on catalysts arranged in stationary or virtually stationary beds in a reactor.

In this process, the catalyst in the reactor is at least partially replaced continuously or at periodic intervals. As used herein, “continuous, at least partial replacement of the catalyst” means that the portion of catalyst being replaced (which must be at least 10% of the catalyst) is permanently discharged from the reactor and replaced with new catalyst that is fed into the reactor. Replacement of the catalyst “at periodic intervals” means that within 20 days after the start of each operating period, at least 10% of the catalyst is replaced, where an operating period is the period of time between two at least partial catalyst replacement operations are carried out, with the initial filling of the reactor being considered as a replacement operation with at least 10% of the catalyst being replaced within 20 days.

In the continuous, at least partial replacement of the catalyst embodiment of the present invention, the total operating time of a reactor is divided into 20 day intervals. It is within each of these 20 day intervals that at least 10% of the catalyst must be replaced. In this embodiment of the present invention, a distinction must be made between continuous replacement of the catalyst and continuous operation of the hydrogenation process.

In the at least partial replacement of the catalyst at periodic intervals embodiment of the present invention, at least 10% of the catalyst is replaced for the first time no later than 20 days after the start of the hydrogenation process with subsequent, at least 10% catalyst replacements occurring no later than 20 days after the previous catalyst replacement.

As used herein, “at least 10% of the catalyst” may mean “% by weight, based on the total weight of the catalyst in the catalyst bed” or “% of the bulk volume, based on the total bulk volume of the catalyst in the catalyst bed”. Generally, from 10 to 100% of the catalyst, preferably from 20 to 90%, more preferably, from 30 to 80%, and most preferably, from 50 to 70% is replaced in the requisite 20 day interval.

In a preferred embodiment of the process, the catalyst removed from the reactor is replaced by catalyst having an activity such that at the latest 24 h, preferably at the latest 12 h, more preferably at the latest 6 h and most preferably at the latest 3 h after changing of the catalyst, an instantaneous selectivity which is at least 99.0%, preferably at least 99.5% and most preferably at least 99.9% of the average selectivity achieved in the last operating period before changing of the catalyst is achieved. As used herein, “operating period” means in each case the time span between two catalyst changing operations.

Preferred nitroaromatics for use in the process of the present invention are those corresponding to the formula

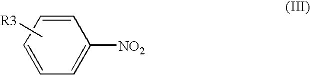

in which R3 has the abovementioned meaning. Nitrobenzene (R3=H) is particularly preferred as the nitroaromatic.

The nitroaromatic can be metered into the reactor as described in DE-OS-1 809 711, but it is preferred that the nitroaromatic be vaporized completely in the fresh hydrogen and then introduced into the circulating gas stream in gaseous form. The advantage of this latter procedure lies in the significantly lower formation of deposits in the reactor and in the feed lines. The vaporization can be carried out by any known procedure using any of the known evaporators, such as falling film, ascending tube, injection, thin film, circulating and coiled tube evaporators. The vaporization can be followed by a mist collection which is known in principle. The educt gas stream is mixed in known manner by an appropriate feeding in and distribution means and/or by mixing devices in the circulating stream.

Atomization of the liquid nitroaromatic into the fresh hydrogen or circulating gas/hydrogen stream by means of one-component or two-component nozzles is also possible. The educt gas stream may be combined with the atomized nitroaromatic after superheating in a heat exchanger.

In the case of the atomization procedure in particular, it has proven to be advantageous to install an additional layer of inert material upstream of the flat catalyst layer in the direction of flow to this layer. This has the advantage that during atomization any non-vaporized drops of the nitro compound employed can be deposited and vaporized further, before they come into contact with the catalyst layer. The catalyst is also protected in this way from any impurities present in the aromatic nitro compound, e.g. high-boiling organic secondary components or salts. Packings of steel wool fabric or bulk aluminum oxide of low BET surface area may be used, for example, as inert materials. In the latter case, the particle diameter of the aluminum oxide is preferably larger compared with the catalyst particles themselves by a factor of 1.5 to 100. The particles used can optionally also be impregnated with an oxidation catalyst, preferably oxide(s) of vanadium. In preferred embodiments, the additional layers of inert materials are arranged such that they can be replaced without interrupting the hydrogenation process.

The process of the present invention can, in principle, be conducted using any desired reactor geometry and procedure. In a particularly economical and therefore preferred embodiment, during the entire hydrogenation process, the activity of the catalyst is kept on average at a level such that the conversion of nitroaromatics, where appropriate, with the exception of a start-up phase at the start of the hydrogenation and brief disturbances in the ideal operating cycle, does not fall below 99.9000% at any point in time.

This is preferably achieved by a process in which
(i) the catalyst is arranged in the reactor in the form of one or more virtually stationary catalyst beds and the catalyst beds are arranged in the reactor in the form of one or more regularly shaped flat catalyst layers (to promote a homogeneous dwell time distribution of the gas flowing through them),
(ii) the removal of catalyst from a flat catalyst layer and the feeding of catalyst into a flat catalyst layer is carried out continuously or at periodic intervals without interrupting the hydrogenation process,
(iii) a gas mixture which contains 3 mol to 150 mol of hydrogen per mol of nitro group flows to the flat catalyst layer or the first of several flat catalyst layers connected in series,
(iv) the hydrogenation is carried out under adiabatic conditions under an absolute pressure of from 1 bar to 50 bar with the entry temperature of the gas mixture employed at from 150° C. to 400° C. and a maximum catalyst temperature of 600° C., and
(v) hydrogen is separated off from the reaction mixture obtained in the hydrogenation and the hydrogen obtained in this way is fed back into the hydrogenation.

This preferred embodiment of the process of the present invention is distinguished in particular in that feeding in and removal of the catalyst can be carried out continuously or at periodic intervals without having to interrupt the hydrogenation process, which is called a migrating bed in this intention.

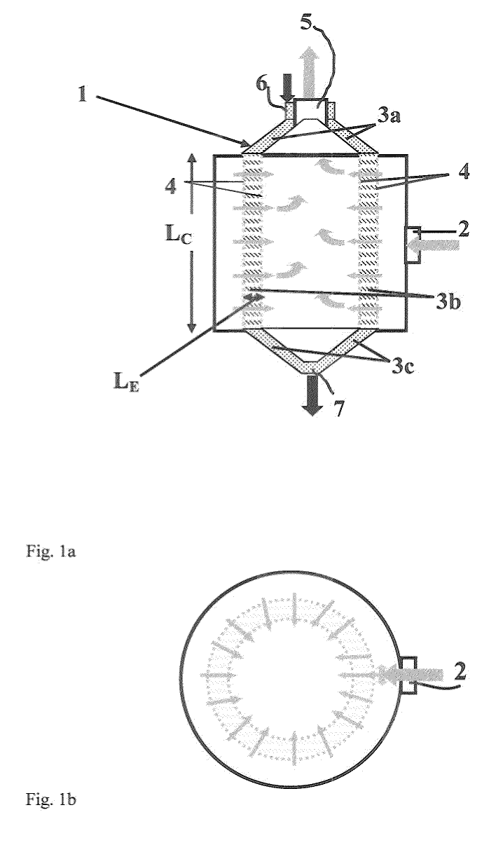

In this preferred embodiment of the process of the present invention, the “migrating bed variant”, only a part of the catalyst in a reactor participates actively, i.e. exerts a catalytic action, in the hydrogenation process at a given point in time (designated 3b in FIG. 1a). Only this catalyst precisely involved actively in the hydrogenation process at a given point in time is arranged in regularly shaped flat layers. A certain portion of the catalyst involved actively in the hydrogenation process is removed continuously or periodically, preferably periodically, from these regularly shaped flat layers and replaced by catalyst fed into the reactor. The amount of catalyst removed, the replacement frequency and the nature of the catalyst fed into the reactor are preferably chosen in this context so that the conversion of nitroaromatics, where appropriate, with the exception of a start-up phase at the start of the hydrogenation and brief disturbances in the ideal operating cycle, does not fall below 99.9000%, preferably below 99.9900%, most preferably below 99.9990%, at any point in time. The hydrogenation process can therefore be operated completely continuously in this embodiment of the present invention, apart from unavoidable interruptions to the production process, for example due to maintenance and inspections prescribed by law. In this embodiment, the expression “at the start of the hydrogenation” accordingly relates to the initial start-up of the reactor and the restarting after such interruptions.

In a particularly preferred embodiment of the migrating bed variant of the process of the present invention, the extension of the flat catalyst layers in the direction of flow of the educt gas mixture (L_{E}) is smaller than the extension in the direction of the catalyst discharge (L_{C}). L_{E} is between 1 cm and less than 100 cm, preferably between 2 cm and less than 50 cm and most preferably between 5 cm and less than 25 cm. In this embodiment, L_{C} is always greater than L_{E} and is preferably not more than 20 m, more preferably not more than 10 m and most preferably not more than 5 m. The direction of flow of the catalyst and the direction of flow of the educt gas mixture are preferably perpendicular to one another, as shown in FIGS. 1a and 1b. Deviations from the ideal right angle in the range of ±20% are also possible.

The flat catalyst layers are preferably installed between gas-permeable walls. Preferred gas-permeable walls are metallic grids or screens in the form of hollow cylinders between which the catalyst is located. Technical devices for uniform distribution of the gas can additionally be installed upstream, downstream or upstream and downstream of the catalyst layers. These can be, for example, perforated plates, bubble trays, valve trays or other installed elements which, by generation of a sufficiently high, uniform pressure loss, have the effect of uniform entry of the gas into the bulk catalyst. As used herein, the descriptions “upstream of the catalyst layer” and “downstream of the catalyst layer” always relate to the direction of flow of the educt gas.

In the process of the present invention, the flat catalyst layers can be arranged in one reactor or in several reactors. A reactor can contain one catalyst layer or several catalyst layers. Several reactors with one catalyst layer can therefore be replaced by a smaller number of reactors with several catalyst layers.

Several catalyst layers in a reactor can be arranged one above the other or side by side. In both cases, all the catalyst layers are preferably equipped with devices for addition and removal of catalyst. If several catalyst layers are arranged one above the other in a reactor, the top catalyst layer is charged with catalyst from outside the reactor, while the remaining catalyst layers are preferably charged with the catalyst which has been removed from the next higher catalyst layer. If several catalyst layers are arranged side by side in a reactor, each is preferably charged with catalyst from outside the reactor.

If several reactors are employed, they can be arranged in series or parallel.

Preferably, at least one reactor is operated adiabatically in the process of the present invention. In this context, preferably not more than 10, more preferably not more than 5, most preferably not more then 3 such reactors are arranged in series. Each of the reactors connected in series can be replaced by several reactors connected parallel. In this context, preferably not more than 5, more preferably not more than 1 most preferably not more than 2 reactors are connected parallel. The process of the present invention accordingly is preferably conducted using not more than 50 and not less than 1 reactor.

The number of catalyst layers in a reactor is preferably between 1 and 10, more preferably between 1 and 5 and most preferably between 1 and 3.

FIGS. 1a and 1b show in diagram form a reactor which is suitable for carrying out the process of the present invention. Sluice devices and similar components have been omitted for clarity. FIG. 1a shows a section in the longitudinal direction and FIG. 1b shows a section in the transverse direction for the same reactor as in FIG. 1a. The reference numbers and symbols used in FIGS. 1a and 1b represent:
1 reactor,
2 educt intake (grey arrows symbolize the flow of the gas mixture),
3a catalyst which can be fed to the hydrogenation process,
3b catalyst participating actively in the hydrogenation process and arranged in a flat-shaped layer,
3c catalyst which can be sluiced out of the reactor,
4 gas-permeable walls,
5 product outlet,
6 catalyst intake (black arrows symbolize the direction of flow of the catalyst),
7 catalyst outlet,
L_{E} the length of the flat catalyst layers in the direction (direction of flow) of the educt gas stream,
L_{C} the length of the flat catalyst layers in the direction of the catalyst discharge.

In the embodiment shown in FIGS. 1a and 1b, the gas mixture first flows from the outside inwards through the catalyst layer and then upwards out of the reactor. Other possibilities for guiding the gas (for example, from the inside outwards and then upwards or from the inside outwards and then downwards or from the outside inwards and then downwards) are likewise conceivable.

The gas mixture is preferably homogenized (for example, mixed in a static mixer) before the start of the hydrogenation on the catalyst layers. A gas mixture which generally contains 3 mol to 150 mol, preferably 6 mol to 125 mol, more preferably 12 mol to 100 mol, most preferably 50 mol to 90 mol of hydrogen per mol of nitro group flows to the flat catalyst layers or the first of several flat catalyst layers connected in series. In a preferred embodiment, the educt gas mixture also contains 0.01 mol to 100 mol, more preferably 3 mol to 50 mol, most preferably 4 mol to 25 mol of water per mol of nitro group. The presence of water in the educt gas stream has proven to be advantageous because it has the consequence of delaying the deactivation of the catalyst due to coking, thereby reducing the frequency of replacement. Water molecules compete successfully with organic molecules for the free centers on the surface of the catalyst, resulting in reduced dwell time of the organic molecules and a slowed down deactivation process.

The gas mixture entering into the reactor has a preferred entry temperature of from 150° C. to 400° C., more preferably 200° C. to 300° C. and most preferably 220° C. to 280° C. Because of the highly exothermic character of the hydrogenation of aromatic nitro compounds, an adiabatic jump in temperature occurs in the catalyst layer. The process parameters are preferably chosen so that temperatures no greater than 600° C., preferably no greater than 550° C. and most preferably not more than 500° C. arise in the catalyst layers.

The hydrogenation is preferably carried out under an absolute pressure of from 1 bar to 50 bar, more preferably 2 bar to 20 bar and most preferably 2 bar to 10 bar. In a preferred embodiment, after leaving a catalyst layer, the reaction mixture is first cooled with the production of vapor (preferably water vapor). This is preferably done by passing the reaction mixture through one or more heat exchangers. These can be any of the heat exchangers known to the person skilled in the art, such as tube bundle, plate, annular groove, spiral flow or ribbed tube heat exchangers. If the reaction mixture is also to flow through further catalyst layers, this cooling preferably reduces the reaction mixture temperature to the entry temperature of the next catalyst layer without condensation of the aromatic amine formed. Preferably, only after flowing through the last catalyst layer is the gas mixture also cooled to the extent that aromatic amine can be removed from the reaction mixture by condensation. In this embodiment, a gas mixture which contains 3 mol to 150 mol of hydrogen and optionally 0.01 mol to 100 mol of water then flows to only the first catalyst layer. The gas mixture which is obtained from the previous catalyst layer and is optionally treated, for example, with fresh hydrogen and fresh nitroaromatic then flows to the next catalyst layer. However, it is also possible to sluice out individual components or to feed in other or further components between the catalyst layers.

In the case of several reactors connected in series, if the aromatic amine is separated off by condensation not only after the last but after each reactor, a gas mixture which contains 3 to 150 mol of hydrogen and optionally 0.01 to 100 mol of water preferably flows to each of the reactors.

Recycling of the hydrogen is preferably carried out by a procedure in which after the condensable constituents of the reaction mixture have been separated off, hydrogen and optionally also inert gas (preferably nitrogen) and optionally water vapor is/are fed back into the hydrogenation process.

The circulating gas stream preferably passes through one or more compressor(s) in order to compensate the flow resistance of reactors and heat exchangers and to control the mass flow of the circulating gas. The compressors can be simple, known machines (e.g., liquid ring pumps, rotary blowers, turbo blowers or turbo compressors), since the pressure loss can be kept small by the construction of the reactors. Dry-running compressors are preferably used.

Preferably, the circulating gas is brought to the entry temperature of from 150° C. to 400° C. again by means of a heat exchanger directly upstream of the first catalyst layer. Upstream or downstream of this heat exchanger, preferably downstream, the nitroaromatic and fresh hydrogen are metered in as described above, and water and inert gas are optionally fed into the reactor.

In a particularly economical variant of the process, the water of reaction obtained in gaseous form is preferably condensed out only incompletely, and the water vapor which remains is fed back together with the remaining circulating gas, so that external addition of water is unnecessary.

The condensate is preferably passed into a technical device for separation of liquid phases, and the aqueous and the organic phase are worked up separately. Aromatic amine obtained from the aqueous phase is fed to the working up of the organic phase. The working up operations are carried out in a known manner by distillation or by stripping with steam. Due to the advantages of the process of the present invention (uniform selectivity at a high level), the working up is simple compared with other processes.

Catalysts which can be employed are in principle any of the contacts described hitherto for the gas phase hydrogenation of nitro compounds. In the preferred embodiment of the migrating bed reactor, it is important that the morphology and mechanical resistance of the catalysts employed allow continuous or periodic feeding in and removal of catalyst. Such catalysts contain, for example, the above-mentioned elements, either as an alloy or as mixed oxides and optionally on an inert support material. Possible support materials are, in particular: α- and γ-Al₂O₃, SiO₂, TiO₂, Fe₂O₃/Al₂O₃ mixtures and CuO/Cr₂O₃ mixtures. However, other supports can in principle also be employed.

In principle, the support materials can have any desired form. In the preferred embodiment of the migrating bed reactor, it is important to ensure that the material is free-flowing. Those support materials of which the morphology is essentially spherical are preferably employed. The sphere diameter of the supports which can be employed in the process of the present invention is between 0.01 mm and 10 mm, preferably between 0.1 mm and 8 mm, more preferably between 0.5 mm and 4 mm and most preferably between 1 mm and 2 mm.

In the preferred embodiment of the migrating bed reactor, the abrasion resistance of the support material is of great importance. Those support materials with a breaking force on average greater than 30 N, preferably greater than 60 N, more preferably greater than 80 N and most preferably greater than 90 N (where the value for the breaking force is a mean of at least 100 measurements on individual spheres (measurement in accordance with DIN EN ISO 604, version of December 2003)) are preferably employed in the process of the present invention.

In the process of the present invention, the BET surface area of the support material is likewise of great importance, because catalysts on supports of very high BET surface area tend towards an increased formation of by-products. Those support materials of which the BET surface area is less than 50 m²/g, preferably less than 25 m²/g, more preferably less than 13 m²/g and most preferably less than 7 m²/g are therefore preferably employed.

Particularly preferred support materials are spheres of α-aluminium oxide with a BET surface area of less than 7 m²/g and a breaking force of greater than 90 N.

The following classes of active substance(s) are preferably precipitated on the support material:
(a) 1-100 g/l_{catalyst} of one or more metals of Groups 8 to 12 of the Periodic Table of the Elements (the designation of the groups of the Periodic Table here and in the following is according to the IUPAC recommendation of 1986), and
(b) 0.01-100 g/l_{catalyst} of one or more transition metals of Groups 4 to 7 and 12 and optionally,
(c) 0.01-100 g/l_{catalyst} of one or more main group elements of Groups 13 to 15.

Elements of Group 12 can therefore optionally act as active substances (a) and (b). Preferred active substances are Pd as metal (a); Ti, V, Nb, Ta, Cr, Mo, W, Re as transition metal (b); and Ga, Pb, Bi as main group elements (c).

The active substances are preferably applied on to the support in the form of their soluble salts, and several treatments (impregnations) per component may he necessary.

It has also proven to be advantageous to dope the catalysts mentioned with a sulfur-containing or phosphorus-containing, preferably phosphorus-containing, compound. Such an additional content of doping agent is preferably 0.001-2% by weight, more preferably 0.01-1% by weight of sulfur or phosphorus, preferably phosphorus, in chemically bonded form, based on the total weight of the catalyst. Preferred phosphorus-containing compounds suitable for doping of the catalysts used in the practice of the present invention are: the oxygen acids of phosphorus H₃PO₄, H₃PO₃, H₃PO₂ or alkali metal salts thereof, such as sodium dihydrogen phosphate, sodium or potassium phosphate or sodium hypophosphite. Possible sulfur-containing compounds are preferably salts of the oxygen acids of sulfur, and the alkali metal salts of sulfuric acid are particularly preferred.

Suitable catalysts include those described in DE-OS 2 849 002 and EP 1 882 681 A1. However, the pretreatment with a base described in DE-OS 2 849 002 is not absolutely necessary.

The loading of the catalysts used in the process of the present invention can be very high can generally be from 0.1 g_{nitroaromatic}/[ml_{catalyst}·h] up to 20 g_{nitroaromatic}/[ml_{catalyst}·h], preferably up to 15 g_{nitroaromatic}/[ml_{catalyst}·h], more preferably up to 10 g_{nitroaromatic}/[ml_{catalyst}·h] and most preferably up to 5 g_{nitroaromatic}/[ml_{catalyst}·h]. In the case of several catalyst layers, the loading can be varied from bulk layer to bulk layer. Preferably, however, the loading is in the range of from 0.1 g_{nitroaromatic}/[ml_{catalyst}·h] up to 20 g_{nitroaromatic}/[ml_{catalyst}·h] in all the catalyst layers. The process of the present invention is accordingly distinguished by high space/time yields.

The preferred migrating bed process embodiment of the process of the present invention allows the removal and introduction of catalyst without having to interrupt the hydrogenation process while it is running.

In this case, the removal of catalyst is preferably carried out by a procedure in which
a uniform flow of educt gas through the catalyst layer is ensured, and/or
the dwell time distribution of the educt gas in the catalyst layer is very narrow, and/or
a homogeneous transportation of the catalyst with a narrow dwell time distribution is achieved, and/or
dead space areas (i.e., dormant or recycling regions of the bulk catalyst in the region through which the educt gas flows) are avoided, and/or
to the greatest extent possible, mechanically gentle transportation of the catalyst is achieved in order to avoid abrasion.

The catalyst is preferably passed into a collecting container or collecting region and discharged from that container or region centrally via a suitable metering discharge. Possible discharge devices include cellular wheel sluices, double flap systems with suitable shut-off organs operated in cycles, such as ball valves, butterfly valves or gate valves, speed-regulated or -adapted screw conveyors, vibration channels or the like.

The transfer from the reaction space into the collecting region, which can be flanged directly to the reactor, must be as symmetrical as possible in construction in order to ensure uniform transportation of solid through all of the reactor regions.

The transition from the reaction chamber into the collecting container can also be effected with tubes which are arranged as equidistantly as possible in one or more rows along the periphery of the flat catalyst layer and which have the same angle of inclination and the same tube length and are arranged symmetrically that lead into the intake region of the collecting container. The tubes are to be arranged on the reactor a sufficient distance below the catalyst layer through which the educt gas flows so that flow of the educt gas through the dead space regions between the tube openings at the base of the reactor is avoided. These dead spaces for the transportation of catalyst are to be minimized, where appropriate, by conical regions in the inflow regions of the individual tubes and/or by corresponding installed elements on the reactor base.

The in- or outflow of the educt or product gas into or out of the inside of the reactor optionally also takes place on the under-side of the reactor. In this case, removal of the catalyst via tubes is advantageous because the gas can be guided in one or more gas tubes which can be arranged between the catalyst collecting tubes.

The feeding in of catalyst is preferably carried out by a procedure in which
a uniform flow of educt gas through the catalyst layer is ensured, and/or
the dwell time distribution of the educt gas in the catalyst layer is very narrow, and/or
a homogeneous transportation of the catalyst with a narrow dwell time distribution is achieved, and/or
dead space areas (i.e., dormant or recycling regions of the bulk catalyst in the region through which the educt gas flows) are avoided, and/or
to the greatest extent possible, mechanically gentle transportation of the catalyst is achieved in order to avoid abrasion.

The catalyst is preferably distributed via a distributing container on the periphery of the flat catalyst layer. The feed into the reactor should be symmetric, e.g. via conical installed elements with an angle of inclination adapted to the properties of the bulk material, in particular the slip angle and angle of repose of the catalyst particles.

The transfer from the distributing container into the flat catalyst layer can also be affected via tubes which are arranged concentrically on the distributing container and open in one or more rows of tubes equidistantly along the periphery of the catalyst layer. The angles of inclination and tube lengths of the individual tubes should coincide.

The inflow region of the catalyst in the reaction space is preferably constructed to have a length such that the catalyst layer has a sufficient height in the region through which the educt gas flows and a bypassing of the educt gas via the gas space above the bulk catalyst is made difficult. This is achieved by long flow paths through the bulk catalyst compared with catalyst layer thickness flowed through.

Feeding into the reactor via individual tubes is particularly suitable if the feed or removal line of the educt or product gas into or out of the inside of the reactor is arranged on the upper side of the reactor. In this case, the gas is guided via one or more gas tubes which can be arranged between the catalyst feed tubes.

The guiding of the catalyst within the region through which the gas flows is preferably conducted in a manner such that
a uniform flow of the gas through the thin, flat catalyst layer with a narrow dwell time distribution is achieved, and/or
the catalyst is transported uniformly and with as little mechanical loading as possible.

The inner and outer jacket of the flat catalyst layer is gas-permeable in construction, e.g. is made of perforated sheet metal, porous material, membranes or, preferably of slotted screens with slots aligned in the direction of flow of the catalyst and an otherwise smooth construction on the catalyst side.

A suitable conveying gas for the catalyst is, for example, hydrogen or mixtures of hydrogen and inert gases or inert gases. The preferred inert gas is nitrogen.

The gas preferably flows through with speeds of from 0.1 m/s to 20 m/s, preferably 0.5 m/s to 10 m/s and most preferably at 1 m/s to 3 m/s. The gas speed is in general not constant because of the change in cross-section of the curved, circular catalyst layer and because of the change in the temperature and composition of the gas in the reaction zone.

The catalyst removed (catalyst discharge) preferably passes through a stripping stage in which the reaction gas is removed from the wedges and the catalyst support particles. Stripping is carried out by means of an inert gas, preferably nitrogen, flowing through.

The dust which may arise as a result of abrasion of the catalyst can be separated off continuously or periodically with any suitable apparatus (e.g., sieves, sifters, cyclones or filters), sluiced out of the process and disposed of.

The catalyst discharge is preferably recycled in a portion by weight of from 1% to 100%, preferably from 70% to 100%, most preferably from 90% to 100%, based on the total weight of the catalyst discharge.

Before feeding back into the reactor, the recycled portion can be regenerated completely or partly by treatment with oxygen-containing gas mixtures, preferably air or air/nitrogen mixtures, at elevated temperature, and in particular at temperatures of from 100° C. to 400° C., preferably at temperatures of from 200° C. to 300° C. This regeneration can be carried out completely (no longer a significant residual carbon content) or also incompletely (e.g., for a shorter time and/or at a lower temperature than in the case of the complete regeneration). The weight content of regenerated catalyst can be 0.1% to 100%, preferably 1% to 50%, most preferably 5% to 30% of the amount of catalyst recycled in total. The regenerated catalyst is optionally mixed with the catalyst portion recycled without regeneration and/or fresh catalyst additionally fed into the reactor for topping up and for compensation of the amount of catalyst sluiced out of the process and/or catalyst worked up externally. This mixing can be carried out by static or moving continuous solids mixers or in discontinuously operating solids mixers.

The catalyst mixture obtained in this way is preferably stripped again before feeding it back into the process in order to remove the oxygen from the wedges and the catalyst support particles. This stripping may be done by flowing an inert gas, preferably nitrogen, through the catalyst.

The catalyst mixture obtained in this way then passes through an activation with an activating gas, preferably hydrogen. The activation is carried out at temperatures of from 100° C. to 400° C., preferably from 200° C. to 300° C.

The catalyst mixture obtained in this way is optionally brought together and mixed with the catalyst recycled without regeneration. These components can also be brought together before the activation, as described above. The catalyst mixture is then fed back into the process.

The catalyst to be fed to the process is transported with a suitable transporting device to the collecting container in the catalyst feed of the reactor. This transportation of solid is preferably effected by conveying fine dust in an inert atmosphere. Nitrogen is preferably used as the inert gas.

The catalyst portion to be regenerated can be sluiced out before or after the transportation of the solid of the catalyst portion which is not to be regenerated, depending on the installation site of the reactors for the regeneration.

The transportation of the solid of the regenerated catalyst portion or catalyst portion to be regenerated can also take place after the separation and before the combination, separately from the recycled catalyst portion which is not to be regenerated.

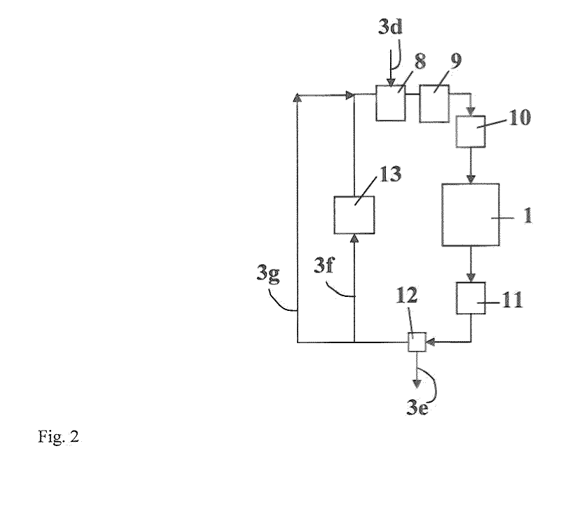

FIG. 2 is a block flow diagram of a possible embodiment of the process of the present invention in which a moving catalyst bed (migrating bed) is employed. The diagram is greatly simplified.

The reference numbers and symbols appearing in Figure represent the following:
1 reactor,
3d catalyst fed to the system from the outside,
3e catalyst dust discharged from the system,
3f catalyst fed to the regeneration,
3g catalyst fed back into the reaction without regeneration,
8 mixing chamber,
9 stripper,
10 activation,
11 stripper,
12 deposition of dust,
13 regeneration.

In a particularly preferred embodiment of the migrating bed variant of the process of the present invention, only that portion of the catalyst removed from the reactor which is to be regenerated is subjected to a stripping stage with inert gas, preferably nitrogen. For safety reasons, the regeneration of this catalyst portion is preferably operated discontinuously in a batch procedure. After the regeneration, the catalyst is stripped again with inert gas and preferably activated with hydrogen. Because of the discontinuous batch procedure of the regeneration, this activation can in principle be carried out in the same apparatus as the regeneration. Preferably, however, an additional apparatus for the activation is connected downstream of the regeneration, which preferably also serves as a storage container for the regenerated and activated catalyst. This catalyst prepared in this way can now be fed to the reaction again. For this purpose, it is mixed completely or partly with the portions fed back without regeneration and optionally the catalyst fed to the system from the outside, which has preferably likewise been activated beforehand by treatment with hydrogen.

Alternatively, regenerated catalyst can also be mixed with catalyst fed in from the outside before the activation, so that a simultaneous activation of the two is possible. This can be realized, for example, by a procedure in which after the regeneration of catalyst already in the system, the regenerated catalyst is first stripped with an inert gas, preferably nitrogen, and then drained completely or partly into a downstream apparatus for activation. Thereafter, catalyst to be fed to the system from the outside is introduced into the regeneration apparatus, which is now under inert gas, and can be drained completely or partly into the activation apparatus. In the activation apparatus, the regenerated and the outside catalyst can then be treated with hydrogen together, before they are fed to the reaction, optionally after admixing with portions of catalyst fed back without regeneration.

Alternatively, catalyst fed in from the outside can also be introduced directly into the activation apparatus, preferably after passing through a stripping stage.

The various catalyst portions (regenerated catalyst and optionally catalyst fed in from the outside and optionally non-regenerated catalyst) are preferably mixed thoroughly before introduction into the hydrogenation reactor, which can be followed by blowing out of catalyst dust, preferably with hydrogen.

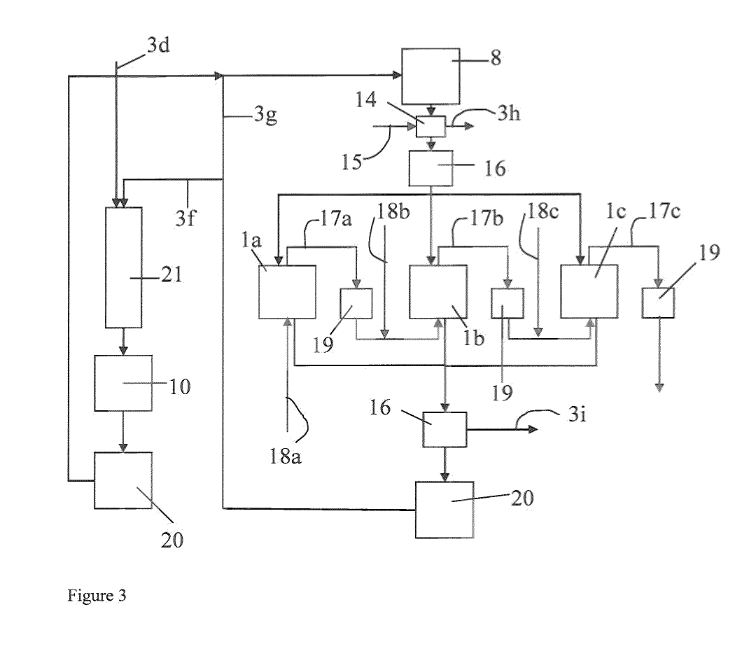

FIG. 3 shows in diagram form a possible block flow diagram of an embodiment of the process of the present invention which is conducted with three reactors. The diagram is simplified for clarity. For example, sluices, valves, circulating gas streams, condensation and working up of the product are not shown. The reference numbers and symbols appearing in FIG. 3 represent the following:
1a, 1b, 1c reactors
3d catalyst fed to the system from the outside
3f catalyst fed to the regeneration
3g catalyst fed into the reaction without regeneration,
3h stream of catalyst dust and hydrogen
3i catalyst discharged from the system
8 mixing chamber
10 activation
14 removal of dust from the catalyst
15 stream of hydrogen for blowing out catalyst dust
16 phased container
17a, 17b, 17c product gas mixture from reactors 1a, 1b, 1c
18a, 18b, 18c educt gas mixture of nitrobenzene, hydrogen and optionally water for reactors 1a, 1b, 1c
19 heat exchanger
20 conveying device for catalyst
21 regeneration and stripping

The process of the present invention leads to the desired amine being formed with a permanently high selectivity which is subject to only slight variations (see examples), which considerably reduces the outlay during working up.

The establishment of a constantly high selectivity subject to only slight variations is ensured in the process of the present invention by the continuous or periodic replacement of the catalyst. Other measures already described earlier for increasing the selectivity, such as partial replacement of the hydrogen employed in a stoichiometric excess by an inert gas (e.g., nitrogen) can also be applied to the process of the present invention, but as a rule these measures are not necessary.

The particular advantage of the preferred migrating bed variant of the process of the present invention compared with the conventional fixed bed process without continuous or periodic replacement of the catalyst lies in the fact that the low selectivities which initially arise with fresh or completely regenerated catalyst according to the prior art and which, as described above, make working up of the product significantly more difficult can be avoided in a simple manner. In the process of the present invention, preferably only small regions of the reactor are operated with catalyst of high activity. There is moreover the possibility of adjusting the activity of the catalyst and therefore also the selectivity of the process to the optimum by the reflux ratio of the catalyst and the content of regenerated catalyst. As a result, a catalyst of moderate activity and optimum selectivity operates in the entire reactor. The average selectivity is also improved in this manner, compared with the prior art processes, and the variations in selectivity are reduced to a minimum. Since in the migrating bed variant of the process of the present invention there is no longer the task of optimizing the service life by improving the catalyst composition, the possibility of even better optimization of the catalyst in the direction of high selectivity arises. Further, production does not have to be interrupted for regeneration of the catalyst.

### Examples

In each of the examples, the catalyst system employed was composed of 9 g/l of Pd, 9 g/l of V and 3 g/l of Pb on α-aluminum oxide (DE-OS-28 49 002). Catalysts of this catalyst system aged to different degrees were employed.
CATALYST A: catalyst already employed in several production cycles, operated to nitrobenzene breakthrough and then regenerated briefly (i.e. treated with air at 290° C. for approx. 10 h).
CATALYST B: catalyst already employed in several production cycles, operated to nitrobenzene breakthrough and then not regenerated.
CATALYST C: catalyst already employed in several production cycles, operated to nitrobenzene breakthrough and then regenerated for a long period of time (i.e. treated with air at 290° C. for approx. 24 h).
CATALYST D: fresh catalyst.

The products were each analyzed by gas chromatography. Percentage data in connection with mixtures of various catalysts are to be understood as meaning % of the bulk volume.

The experiments described below in Examples 1 and 2 were carried out in a thermostatically controlled tube reactor which in each case contained a bulk volume of 100 ml of catalyst. The temperature of the heat transfer medium was increased stepwise from 250° C. to 300° C. at a rate which was the same in all of the experiments. Nitrobenzene was reacted to give aniline at a molar ratio of hydrogen to nitrobenzene of approximately 12.5:1.

### Example 1

### According to the Invention, Fixed Bed Variant—Interruption of the Hydrogenation for Changing of the Catalyst

The reactor was filled with a homogeneous mixture of 20% of CATALYST A and 80% of CATALYST B. Nitrobenzene was fed in for as long as the conversion did not fall below 99.9900% (130 h; 10.95 kg of nitrobenzene in total; operating period I).

First Change of Catalyst and Next Operating Period:

The catalyst was removed completely and replaced by a homogeneous mixture of 40% of CATALYST A and 60% of CATALYST B. Nitrobenzene was fed in for as long as the conversion did not fall below 99.9900% (134 h; 11.55 kg of nitrobenzene in total; operating period II).

Second Change of Catalyst and Next Operating Period:

The catalyst was removed completely and replaced 100% by CATALYST A. Nitrobenzene was fed in for as long as the conversion did not fall below 99.9900% (184 h; 15.46 kg of nitrobenzene in total; operating period III).

Third Change of Catalyst and Next Operating Period:

The catalyst was removed completely and replaced by a homogeneous mixture of 60% of CATALYST A and 40% of CATALYST B. Nitrobenzene was fed in for as long as the conversion did not fall below 99.9900% (150 h; 12.70 kg of nitrobenzene in total; operating period IV).

Fourth Change of Catalyst and Next Operating Period:

The catalyst was removed completely and replaced by a homogeneous mixture of 80% of CATALYST A and 20% of CATALYST B. Nitrobenzene was fed in for as long as the conversion did not fall below 99.9900% (155 h; 12.47 kg of nitrobenzene in total; operating period V).

### Example 2

### Comparison Example—No Change of Catalyst, Interruption of the Hydrogenation for Regeneration in the Reactor

The reactor was filled completely with CATALYST C. Nitrobenzene was fed in for as long as the conversion did not fall below 99.9900% (342 h; 31.57 kg of nitrobenzene in total; operating period 1).

Regeneration and Next Operating Period:

The spent catalyst was not removed but regenerated in the reactor with air at 290° C. for 24 h. Thereafter, nitrobenzene was fed in again for as long as the conversion did not fall below 99.9900% (341 h; 30.35 kg of nitrobenzene in total; operating period II).

The procedure described in Example 2 corresponds to the current prior art in fixed bed reactors.

The following table shows a comparison of the results obtained in Examples 1 and 2.

TABLE 1
Comparison of the results from Examples 1 and 2

| | Operating | | S_{I}/% ^{[a]} | | | | | | |
|---|---|---|---|---|---|---|---|---|---|
| Ex. | period | Duration/h | t = 24 h | t = 48 h | t = 72 h | t = 96 h | t = 120 h | ΔS_{I} ^{[b]} | S_{A}/% ^{[c]} |
| 1 | I | 130 | 99.93 | 99.94 | 99.94 | 99.94 | 99.94 | 0.01 | 99.94 |
| | II | 134 | 99.93 | 99.94 | 99.93 | 99.94 | 99.94 | 0.01 | 99.94 |
| | III | 184 | 99.60 | 99.84 | 99.90 | 99.92 | 99.92 | 0.32 | 99.85 |
| | IV | 150 | 99.89 | 99.93 | 99.94 | 99.94 | 99.94 | 0.05 | 99.92 |
| | C | 155 | 99.87 | 99.92 | 99.93 | 99.93 | 99.94 | 0.07 | 99.91 |
| | Entire experiment | | | | | | | | 99.91 |
| 2 | I | 342 | 95.30 | 98.50 | 99.24 | 99.46 | 99.60 | 4.30 | 99.42 |
| | II | 340 | 96.05 | 98.82 | 99.37 | 99.61 | 99.70 | 3.65 | 99.55 |

| | |
|---|---|
| Entire experiment | 99.49 |

Explanations:
^{[a]} S_{I}: Instantaneous selectivity at time t.
^{[b]} ΔS_{I} = [S_{I}(120 h)/%] − [S_{I}(24 h)/%].
^{[c]} S_{A}: Average selectivity in the stated operating period or in the entire experiment.

At comparable total amounts of nitrobenzene employed and the same requirements regarding minimum conversion, significantly lower average selectivities were achieved in the comparative Example 2 than in Example 1. The variations in selectivity are lower in Example 1 than in the comparative Example 2 by one order of magnitude.

### Example 3

### According to the Invention, Migrating Bed Variant

This experiment was carried out by a circulating gas procedure in an adiabatically operated reactor. The reactor offered the possibility of feeding in and removing the catalyst via a sluice system without interrupting the hydrogenation process. An incorporated stirring system moreover allowed thorough mixing of the catalyst also during the reaction. Nitrobenzene was reacted to give aniline at an entry temperature of the educt gas mixture of approximately 240° C., an absolute pressure of 4 bar, a loading of 1.0 g_{nitrobenzene}/ml_{cat.}·h) and a molar ratio of hydrogen to nitrobenzene of approx. 80:1. The reactor was filled with a bulk volume of approximately 530 ml of a homogeneous mixture of 20% of CATALYST B (different batch than that used in Example 1) and 80% of CATALYST C (different batch than that used in Example 2), corresponding to a bed height of 20 cm. At certain times in each case, approximately 30 ml of catalyst were removed from the reactor and replaced by other catalyst. In some cases, stirring was carried out in each case for 2 minutes after addition of the catalyst, in order to homogenize the bulk catalyst. Details are contained in Table 2.

TABLE 2
Conditions in Example 3

| | Change of cat. after | | | | | | | | | | | |
|---|---|---|---|---|---|---|---|---|---|---|---|---|
| | 18 h | 21 h | 24 h | 42 h | 45 h | 48 h | 66 h | 72 h | 90 h | 93 h | 113 h | 137 h |
| Catalyst | B/C | B/C | B/C | B/C | B/C | B/C | B/C | B/C | B/C | B/C | B/C | B/C |
| added | (20/80) | (20/80) | (20/80) | (20/80) | (20/80) | (20/80) | (20/80) | (20/80) | (20/80) | (20/80) | (20/80) | (20/80) |
| (mixture | | | | | | | | | | | | |
| ratio) | | | | | | | | | | | | |
| Stirred? | no | no | no | yes | yes | yes | yes | yes | yes | yes | yes | yes |
| | Change of cat. after | | | | | | | | | | | |
| | 161 h | 165 h | 185 h | 209 h | 255 h | 300 h | 347 h | 371 h | 395 h | 419 h | 443 h | 467 h |
| Catalyst | B/C | B/C | B/C | B/C | B/C | B/C | B/C | B/C | B/C | B/C | B/C | B/C |
| added | (20/80) | (20/80) | (10/90) | (10/90) | (10/90) | (10/90) | (10/90) | (10/90) | (10/90) | (10/90) | (10/90) | (10/90) |
| (mixture | | | | | | | | | | | | |
| ratio) | | | | | | | | | | | | |
| Stirred? | yes | yes | yes | yes | yes | yes | yes | yes | yes | yes | yes | yes |

The experiment was interrupted after 491 hours; however, continuation would have been easily possible. The average nitrobenzene content in the product was 86 ppm.

This example shows that the replacement of catalyst already demonstrated in Example 1 can also be carried out without interrupting the hydrogenation process, which represents an enormous advantage for large-scale industrial use. The average nitrobenzene content in the product of 86 ppm is still in the acceptable range.

### Example 4 (Comparison Example)

This experiment was carried out in a three-stage adiabatically operated reactor with a circulating gas system. The reactors were each filled with a bulk volume of approx. 540 ml of CATALYST D, corresponding to a bed height of 20 cm. The experiment was started with a specific loading in the first reactor of 1.0 g_{nitrobenzene}/(ml_{cat}·h), which was increased stepwise in the course of the experiment in order to achieve better selectivities (after 24 h to 1.5 g_{nitrobenzene}/(ml_{cat.}·h) and after 48 h to 2.7 g_{nitrobenzene}/(ml_{cat.}·h). A molar ratio of hydrogen to nitrobenzene of approximately 80:1 was employed. The absolute pressure was 4 bar, the entry temperature of the educt gas mixture was about 240° C. The procedure corresponds to the current prior art for an adiabatic process procedure. The experiment was carried out for 312 h. Nitrobenzene breakthrough was then observed in the sampling point after the third reactor. Until then, the average content of nitrobenzene in the product after the first reactor was 2 ppm.

The installation allowed sampling after each reactor. For the comparison with Example 3, only the analytical results after the first reactor were used.

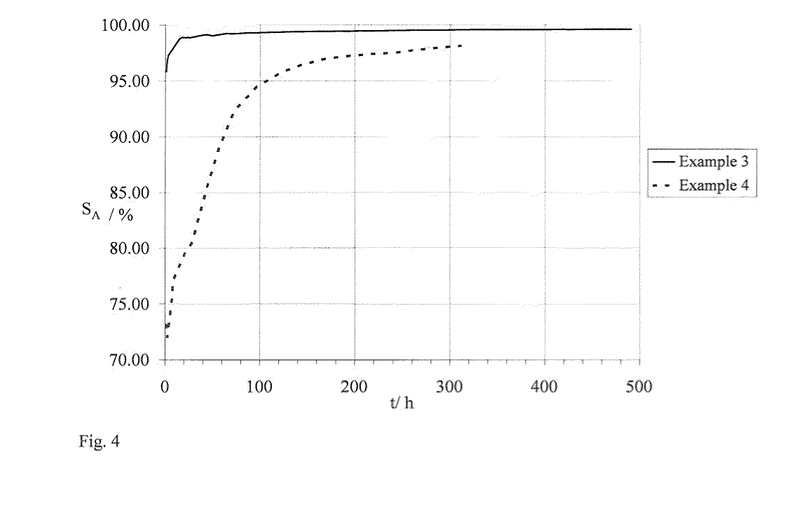

FIG. 4 shows the average selectivities S_{A} achieved in Example 3 and 4 in comparison with one another. (The value for S_{A}(t) at a given point in time t designates the average selectivity with which the total aniline obtained up to this point in time was prepared.) The values in Example 4 are those after the first reactor of the three-stage installation.

The aniline was formed with a significantly higher selectivity in Example 3 than in Comparison Example 4. Although the specific loading was increased in Example 4 in order to improve the selectivity (reduction in the dwell time of the aniline formed on the catalyst), it was possible to achieve only significantly poorer results than in Example 3. The improvement in selectivity in Example 3 compared with comparative Example 4 is so great that the disadvantage of the higher nitrobenzene content in the product is over-compensated. The migrating bed variant from Example 3 allows replacement of the catalyst without interrupting the hydrogenation process, so that the activity of the catalyst involved actively in the hydrogenation process varies only within narrow limits, and in particular at a level such that high selectivities result therefrom from the start.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of an aromatic amine of the formula 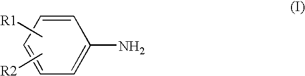
in which
R1 and R2 independently of each other represent hydrogen, a methyl group or an ethyl group, and
R1 can also represent an amino group,
comprising reacting a nitroaromatic compound of the formula 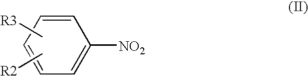
in which
R2 and R3 independently of each other represent hydrogen,
a methyl group or an ethyl group, and
R3 can also represent a nitro group,
in the gas phase with hydrogen over a catalyst arranged in stationary or virtually stationary beds in a reactor in which at least 10% of the catalyst is replaced within each 20 day interval subsequent to start up or the start up of an operating period of the reaction.

2. The process of claim 1 in which
(a) the catalyst is arranged in the reactor in the form of one or more virtually stationary catalyst beds,
(b) the one or more catalyst beds are arranged in the reactor in the form of one or more regularly shaped flat catalyst layers,
(c) removal of catalyst from a flat catalyst layer and feeding of catalyst into a flat catalyst layer are carried out continuously or at periodic intervals without interrupting the reaction of the nitroaromatic compound with hydrogen,
(d) a gas mixture which contains 3 mol to 150 mol of hydrogen per mol of nitro group flows to a first flat catalyst layer,
(e) the reaction of the nitroaromatic compound and hydrogen is carried out under adiabatic conditions under an absolute pressure of from 1 bar to 50 bar at an entry temperature of the gas mixture employed of from 150° C. to 400° C. and a maximum catalyst temperature of 600° C.,
(f) hydrogen is separated off from the aromatic amine-containing reaction mixture and the separated hydrogen is recycled to react with nitroaromatic compound.

3. The process of claim 2 in which the gas mixture's direction of flow is essentially perpendicular to catalyst discharge direction.

4. The process of claim 3 in which the flat catalyst layer's length with respect to direction of incoming educt gas stream_{E} L_{E} is shorter than the flat catalyst's length with respect to direction of catalyst discharge L_{C}.

5. The process of claim 4 in which L_{E} is between 1 cm and less than 100 cm and L_{C} is not more than 20 m.

6. The process of claim 5 in which a gas mixture comprising from 3 mol to 150 mol of hydrogen and from 0.01 mol to 100 mol of water per mol of nitro group flows to the flat catalyst layer or the first of several flat catalyst layers connected in series.

7. The process of claim 2 in which a gas mixture comprising from 3 mol to 150 mol of hydrogen and from 0.01 mol to 100 mol of water per mol of nitro group flows to the flat catalyst layer or the first of several flat catalyst layers connected in series.

8. The process of claim 5 in which (i) the catalyst removed from the reactor the total weight of the catalyst removed in an interval of time and
(ii) before feeding back into the reaction in portions by weight of from 0.1% to 100%, based on the total weight of the catalyst to be fed back into the hydrogenation, at least a portion of the catalyst removed from the reactor is regenerated with an oxygen-containing gas mixture at a temperature between 100° C. and 400° C. and
(iii) non-regenerated portions of the catalyst removed from the reactor are fed back into the reaction together with the regenerated portions of the catalyst removed from the reactor.

9. The process of claim 2 in which
(i) the catalyst removed from the reactor is fed back into the reaction in portions by weight of from 1% to 100%, based on the total weight of the catalyst removed in an interval of time and
(ii) before feeding back into the reaction in portions by weight of from 0.1% to 100%, based on the total weight of the catalyst to be fed back into the hydrogenation, at least a portion of the catalyst removed from the reactor is regenerated with an oxygen-containing gas mixture at a temperature between 100° C. and 400° C. and
(iii) non-regenerated portions of the catalyst removed from the reactor are fed back into the reaction together with the regenerated portions of the catalyst removed from the reactor.

10. The process of claim 9 in which regeneration of the catalyst removed from the reactor is carried out only incompletely.

11. The process of claim 8 in which regeneration of the catalyst removed from the reactor is carried out only incompletely.

12. The process of claim 9 in which the catalyst is homogenized before or after being fed back into the reactor:

13. The process of claim 8 in which the catalyst is homogenized before or after being fed back into the reactor.

14. The process of claim 2 in which abraded catalyst material is discharged continuously or periodically from the process and replaced by feeding in catalyst from a supply outside of the reactor.

15. The process of claim 1 in which nitrobenzene or nitrotoluene is employed as the nitroaromatic.

16. The process of claim 1 in which the catalyst employed comprises:
(a) from 1 to 100 g/l_{catalyst} of palladium and from 0.01 to 100 g/l_{catalyst} of vanadium or
(b) from 1 to 100 g/l_{catalyst} of palladium and from 0.01 to 100 g/l_{catalyst} of vanadium and from 0.01 to 100 g/l_{catalyst} of lead or
(c) from 1 to 100 g/l_{catalyst} of palladium and from 0.01 to 100 g/l_{catalyst} of vanadium and from 0.01 to 100 g/l_{catalyst} of gallium on a support of α-aluminium oxide with a BET surface area of less than 50 m²/g and a breaking force of greater than 30 N.
